(19) United States
(12) Patent Application Publication
(10) Pub. No.: US 2024/0325151 A1
(43) Pub. Date: Oct. 3, 2024

(54) PERSONALIZED IMPLANT

(71) Applicants: Blok Additive Manufacturing B.V., Velsen-Noord (NL); Stichting Het Nederlands Kanker Instituut - Antoni van Leeuwenhoek Ziekenhuis, Amsterdam (NL)

(72) Inventors: Boris Theodore van Putten, Zoeterwoude (NL); Munir Baris Karakullukcu, Leiden (NL); Robert van Veen, 's- Gravenhage (NL)

(73) Assignees: Blok Additive Manufacturing B.V., Velsen-Noord (NL); Stichting Het Nederlands Kanker Instituut - Antoni van Leeuwenhoek Ziekenhuis, Amsterdam (NL)

(21) Appl. No.: 18/580,629

(22) PCT Filed: Jul. 20, 2022
(86) PCT No.: PCT/NL2022/050426

§ 371 Date: Jan. 19, 2024

(30) Foreign Application Priority Data
Jul. 21, 2021 (NL) 2028801

(51) Int. Cl. A61F 2/28; A61F 2/30

(52) U.S. Cl. CPC A61F 2/2803; A61F 2/30942; A61F 2002/30593

(57) ABSTRACT

A personalized implant for bone replacement comprises an implant body as a reconstruction of a bone portion. The implant body comprises a closed cavity and a solid impervious layer of material defining an outside surface of the implant body exposed to an exterior of the implant. The solid impervious layer fully encloses the closed cavity inside the implant body. The closed hollow interior can comprise an open support structure to reinforce the solid outside layer.

### FIELD OF THE INVENTION

The invention relates to a personalized implant and a method of manufacturing a personalized implant.

### BACKGROUND OF THE INVENTION

In anatomy, the mandible, lower jaw or jawbone is the largest, strongest and lowest bone in the human facial skeleton. It forms the lower jaw and holds the lower teeth in place. The mandible sits beneath the maxilla. It is the only movable bone of the skull, apart from the ossicles of the middle ear. It is connected to the temporal bone by the temporomandibular joint. In case a tumor occurs in or invades the mandible, a surgical resection of the mandible bone containing the tumor may be necessary. Also in other situations, such as trauma, it may be necessary to reconstruct a segment of the mandible. In view of the important functions of the mandible for e.g. chewing, but also in view of the esthetic impact of the mandible, the mandible may be reconstructed after the resection. For example, a bone graft or a flap may be harvested and used to reconstruct the mandible. Such bone grafts are known to be supported by implants, such as metal strips that support the bone graft and re-connect the remaining mandible segments with the bone graft. Implants may be made of a biocompatible material, such as titanium alloy. Using additive manufacturing, patient-specific implants can be made. However, mandibular reconstruction remains a challenging procedure. In practice, the result is often less than optimal. Moreover, the surgical procedure and implant can lead to medical complications, in which case it may even be necessary to perform additional surgery.

US 2017/014169 A1 discloses devices for musculoskeletal reconstructive surgery. The document describes a device used in conjunction with fixation hardware to provide a two-stage process to address the competing needs of immobilization and re-establishment of normal stress-strain trajectories in grafted bone.

However, problems remain and it would be advantageous to further improve the techniques for mandibular reconstructions. Although the above discussion focuses on mandibular reconstruction, the techniques disclosed herein may also be applied to implants for other human or animal bones and organs.

### SUMMARY OF THE INVENTION

It is an object of the present invention to provide an improved implant. The problem is solved by providing a personalized implant for bone replacement. The implant comprises an implant body comprising a closed cavity and a solid impervious layer of material defining an outside surface of the implant body exposed to an exterior of the implant, the solid impervious layer fully enclosing the closed cavity inside the implant body.

The personalized implant may have the advantage that it is less prone to infections because there is no fluid exchange between the closed cavity and the exterior of the implant. This way, the closed hollow interior does not come into contact with the tissues and bone of the patient. The construction may allow to better control the overall mass of the implant by determining the dimensions of the outside layer and the closed cavity.

The closed cavity may comprise an open support structure to reinforce the solid impervious layer. This improves the strength of the implant and allows to further control and reduce the mass of the implant.

The outside surface of the implant body may be piecewise smooth. This may further prevent infections or irritations.

The outside surface may comprise at least one planar section. A planar section may be used to fit the implant to a resection surface of the remaining bone of the patient. For example, the outside surface comprises at least two planar sections, arranged on opposite ends of the implant body. This allows to fit the implant body in between two remaining bone sections, by aligning the planar sections with cutting planes of the bone.

The implant may further comprise at least one flap extending from the implant body at an edge of the at least one planar section of the outside surface. The at least one flap may help to fix the implant into the remaining bone of the patient in a stable manner.

The at least one flap may have a concave surface. This way the flap is reinforced by its shape.

At least one of the flaps may comprise at least three screw holes arranged in a pattern of at least one triangle. This pattern provides for improved stability of the fixation of the implant to the bone.

A weight of the implant divided by a volume of the implant may be at most 2.3 grams per cubic centimeter, and a density of the solid layer may be at least 4 grams per cubic centimeter. This may allow a particularly sturdy material for the solid layer, while the weight of the implant approaches the natural anatomical weight of the resected bone. This provides more comfort for the user of the implant.

The implant may further comprise an outside mesh structure on the outside surface, wherein the outside mesh structure is exposed to an exterior of the implant. This may facilitate osseointegration in certain cases.

According to another aspect of the invention, a method of producing a personalized implant for bone replacement is provided, the method comprising
determining a shape of an implant body based on an anatomical shape of a bone of a patient;
creating the implant body according to the determined shape,
wherein the implant body comprises a closed cavity and a solid impervious layer of material defining an outside surface of the implant body exposed to an exterior of the implant, the solid impervious layer fully enclosing the closed cavity inside the implant body.

The method allows to provide a personalized bone replacement implant of which the shape and weight may be controlled and that that minimizes the risk of irritations or infection, because the closed cavity is fully closed so that it is not exposed to infections from outside.

The closed cavity may comprise an open support structure to reinforce the solid impervious layer. This may help to make the implant body sturdier and longer lasting after implant.

The creating the implant body may comprise creating the solid impervious layer with at least one hole in the solid impervious layer, wherein the at least one hole fluidly connects the cavity with an exterior of the implant body. Further, the method may comprise removing feedstock material from the cavity using the at least one hole; and sealing the at least one hole to fluidly close the cavity. This is an efficient way to create an implant body with a clean hollow interior. For example at least two holes may be created: at least one hole as air inlet and at least one hole as outlet for air and feedstock material.

The determining the shape of the implant body may comprise obtaining a shape model of the bone of the patient; determining at least one cutting plane for resecting the bone; and determining the shape of the implant body based on the shape model and the at least one cutting plane. In doing so, the outer surface of the solid layer of the implant body may comprise at least one planar section corresponding to the at least one cutting plane. This allows the implant body to be an accurate fit to the removed section of bone.

The method may comprise creating at least one flap extending from the implant body at an edge of the at least one planar section corresponding to the at least one cutting plane. This allows the implant body to be fixated to the remaining bone in a stable manner.

The method may comprise determining, based on the shape model, a curvature of the at least one flap to match a curvature of a surface of the bone of the patient adjacent the at least one cutting plane. This provides an improved stability of the implant. The method may comprise creating at least three screw holes in the at least one flap, wherein the at least three screw holes are arranged in a pattern of at least one triangle. This provides an improved distribution of forces to which the implant is subjected, thereby improving the stability of the implant.

The method may comprise creating a cutting jig having at least one cutting guide and at least one flap, wherein the at least one cutting guide defines the at least one cutting plane and wherein a shape, orientation, and location of the at least one flap of the at least one cutting jig with respect to the at least one cutting plane matches at least part of a shape, an orientation, and a location of the at least one flap extending from the implant body. This provides the ability to precisely position the jig in the correct anatomical place on the bone and perform pre-drilling of the holes that are necessary to fix the implant to the bone.

The method may further comprise creating screw holes in the flap of the cutting jig matching corresponding screw holes in the flap of the implant. The screw holes of the cutting jig may be provided with drilling guides. By using the same locations for the screws for the flaps of both the jig and the implant, the number of holes made in the patient's bone may be reduced.

The person skilled in the art will understand that the features described above may be combined in any way deemed useful. Moreover, modifications and variations described in respect of the implant may likewise be applied to the method, and modifications and variations described in respect of the method may likewise be applied to the implant.

### BRIEF DESCRIPTION OF THE DRAWINGS

In the following, aspects of the invention will be elucidated by means of examples, with reference to the drawings. The drawings are diagrammatic and may not be drawn to scale. Throughout the drawings, similar items may be marked with the same reference numerals.

FIG. 1 shows a first perspective view of an example of a mandibular implant.

FIG. 2 shows a second perspective view of the mandibular implant.

FIG. 3A shows a first section view of the mandibular implant.

FIG. 3B shows the first section view of the mandibular implant, during a production stage.

FIG. 4 shows a second section view of the mandibular implant.

FIG. 5 shows a first perspective view of second example of a mandibular implant.

FIG. 6 shows a second perspective view of the second example mandibular implant.

FIG. 7 shows a diagram of a slot for a dental implant.

FIG. 8A shows a front view of a cutting jig.

FIG. 8B shows a top view of the cutting jig.

FIG. 8C shows a back view of the cutting jig.

FIG. 9A shows a flowchart illustrating a method of manufacturing a personalized implant.

FIG. 9B shows a flowchart illustrating a method of determining a shape of an implant body.

FIG. 9C shows a flowchart illustrating a method of designing a flap of an implant.

FIG. 9D shows a flowchart illustrating a method of manufacturing a personalized cutting jig.

FIG. 10A shows a first view of a personalized mandibular implant fixed to a bone.

FIG. 10B shows a second view of the personalized mandibular implant fixed to the bone.

### DETAILED DESCRIPTION OF EMBODIMENTS

Certain exemplary embodiments will be described in greater detail, with reference to the accompanying drawings.

The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail, since they would obscure the description with unnecessary detail.

According to certain embodiments, a personalized implant for bone replacement may comprise an implant body with a solid layer of material enclosing a closed cavity inside the implant body. The material of the solid layer comprises at least one layer of an impervious material, i.e. a material that is not transmissive for gases or liquids, in particular air or bodily fluids. Also, the solid layer may comprise at least one layer of a rigid material, so that the implant body does not deform easily. For example, the solid layer may be made of a homogeneous material or laminated. For example, on the outside, the solid layer may comprise a coating.

The solid layer may form an outside surface exposed to the exterior of the implant, wherein the solid layer fluidly closes the closed cavity. That is, there is no fluid exchange between the closed cavity and the exterior of the implant.

In certain embodiments, the outside surface of the implant body is piecewise smooth. For example, the implant body comprises a surface area with a curved shape to simulate a natural bone shape. Moreover, the outside surface of the implant body may comprise at least one planar section. This planar section, usually at least two planar sections, may correspond to the cutting planes of the bone resection. The implant body may be shaped based on dimensions of the resected bone and the distance and relative orientation of the cutting planes may be represented by the planar sections of the implant body.

In certain embodiments, the implant body or the entire implant is made by means of additive manufacturing.

In certain embodiments, the closed cavity comprises a support structure to reinforce the solid layer. For example, the support structure may comprise a mesh and/or one or more columns, beams or walls of a solid material, inside the closed cavity, connecting one side of the closed cavity with the other side of the closed cavity. This support structure is preferably an open support structure, i.e. a support structure that does not by itself contain any further closed cavities, so that the inner cavity is one connected inner space. For example, in case one or more holes are created in the solid layer during manufacturing, the cavity may be cleaned by applying air pressure through the holes. The open support structure allows the air to pass, e.g. from one hole to the other hole. Further, the weight of the implant body may be controlled by varying the amount of material used in the support structure and/or the solid layer.

The support structure may be generated by means of additive manufacturing while creating the implant body. Alternatively, a support structure may be prepared and subsequently the solid layer may be applied around it afterwards. In certain embodiments, the support structure comprises the same material as the solid layer. In certain embodiments, the support structure inside the closed cavity is or comprises a mesh structure. The mesh structure may comprise a rectangular 3D mesh, a triangular 3D mesh, or any other suitable reinforcing mesh structure. For example, the mesh may be implemented by thin rods arranged in a pattern, such as a rectangular or triangular pattern.

In certain embodiments, the implant body 101 may comprise a plurality of such closed cavities.

At least one flap may extend from the implant body at an edge of the at least one planar section of the outside surface of the implant body. This flap may be curved to improve the strength thereof. Moreover, the shape and orientation of the surface of the flap may match the shape of the remaining bone of the patient near the cutting plane, so that the flap may fit tightly on the bone of the patient. For example, the surface on one side of the flap faces the planar section. The surface on the other side of the flap faces away from the implant. At least near the implant body, the general orientation of the flap may be aligned with the outside surface of the implant body adjacent the rigid section on the side of the flap facing away from the implant. More particularly, the surface of the flap that faces the planar section may be in line with the outside surface of the implant body on the side of the flap opposite the planar section. The construction of such planar sections and rigid flaps extending from the edge of the planar sections helps to clamp the implant in the intended place between the cutting planes. A concave surface of a flap may help to reinforce the rigidity of the flap and better distribute any forces over the several screws with which the implant may be attached to the bone. For example, the flaps may comprise at least three screw holes each, arranged in a non-linear pattern. This further enhances the distribution of forces over the screws compared to a linearly arranged screw pattern.

In certain embodiments, the implant further comprises an outside mesh structure on an outside of the solid layer, exposed to an exterior of the implant. Alternatively, in certain embodiments, at least part of the outside surface of the implant may be rough or porous. This way, osseointegration may be realized. Alternatively, in certain embodiments the entire outside surface of the implant or implant body may be impervious and piecewise smooth.

In certain embodiments, the implant is a bone replacement. Such an implant may comprise a hollow implant body, which largely may serve as a reconstruction of the bone portion that the implant serves to replace. Moreover, the implant may comprise a fixation means to fix the implant body in its proper place. In certain embodiments, the fixation means may comprise one or more flaps extending from the implant body, wherein the flaps can be fixed to the remaining bone of the patient. In certain embodiments, the flaps may comprise holes for screws or pins, so that the flaps may be attached to the remaining bone by means of one or more screws or pins. In certain embodiments, the arrangement of cutting planes and flaps is such that the implant is clamped in between the cutting planes of the remaining bone.

In certain embodiments, the implant body is designed as a hollow object with an optional internal support structure, such as a mesh structure. This structure may result in a relatively light, yet strong implant body. In certain embodiments, the amount of material used for the implant body can be chosen so that the weight of the implant is relatively close to the weight of a removed portion of anatomical bone, while still fully filling up the space of the removed bone portion.

In certain embodiments, 3D printing, or additive manufacturing, is used to create at least part of the implant based on a patient specific design. In certain embodiments, the additive manufacturing is used to print the entire implant, and optionally a patient specific cutting jig or sawing jig.

In certain embodiments, at least one hole, preferably at least two holes, are created in the implant. For example, these holes may be included in the design that is the basis of the additive manufacturing process. The at least one hole may be used to remove any feedstock, e.g. any remaining printing powder residue or dust, from the interior of the implant body, after the additive manufacturing. In case at least two holes are used, this process may be performed more efficiently by means of e.g. air pressure flushing or fluid pressure flushing, in particular liquid pressure flushing. After this, the hole(s) may be closed to seal the inner space from the exterior of the implant body. This closing may be performed, for example, by welding. After closing, the well spot location may be smoothened so that it forms a smooth surface with the remainder of the outside surface of the implant body. Afterwards, the implant body may be polished.

The implant thus created may allow to create a better reconstruction with better esthetics. Moreover, by virtue of the improved distribution of forces and/or the reduced weight, there may be reduced likelihood that the implant impairs or compromises the surrounding tissues.

In certain embodiments, the implant body may be kept stably in position because it fits very well in the space that is created by the removed portion of the bone. In certain embodiments, the surface of the implant body is shaped to match the shape of the resection cut in the bone. This improves the stability of the implant. Rigid flaps may allow to clamp the implant even more stably in the gap between two resection planes. In certain embodiments, the implant body may be fixed in place by, for example, one or more flaps.

In certain embodiments, the implant may comprise at least one flap. This flap may be rigid and may be shaped to match a shape of the bone adjacent the removed portion of the bone. For example, the shape of the flaps may be slightly concave. This makes the flaps less vulnerable to bending. The flaps may have screw holes, so that they can be attached to the bone by screws. Since the flaps do not bend easily, forces exerted on the implant are better distributed over the screws. This may reduce the probability that a screw becomes loose, even after repetitive load bearing stress, which may be caused by for example chewing.

In existing strip type implants, the screw closest to the resection may be exposed to relatively large forces, so that it may fail more often. This drawback may be overcome by the features of the concave shape that does not bend easily and the arrangement of the screws. For example, the width of the flaps may be sufficient to allow multiple screws arranged in a triangular (i.e. two-dimensional) pattern. This pattern of screw fixations may further enhance the stability of the implant, and/or improve resistance against torsion and bending.

In certain embodiments, the implant is a mandibular implant to replace a segment of the mandible. In certain embodiments, the implant body may comprise integrated dental implants. This facilitates dental reconstruction.

A cutting jig may be constructed by additive manufacturing, e.g. 3d printing. A cutting jig may comprise one or more cutting guides and a structure to control the orientation of the cutting guides with respect to the bone to be cut. The cutting jig may also comprise drilling guides to facilitate controlled drilling of holes and/or placement of screws. However, during use of the cutting guide, in particular when the sawblade is operated inside the cutting guide and touches the material of the cutting guide, or when a drill is operated inside a drilling guide, sawdust of the material of the cutting guide may be generated as an undesirable side effect. In certain embodiments, the cutting jig is made of a metal to make the cutting more stable and accurate. Also, the material of a metal cutting jig may be strong enough to prevent sawdust of the material of the jig to be generated during cutting and possibly unintendedly ending up inside the patient. Also, a metal cutting jig may be less prone to bending, which further improves the accuracy of the intended drilling holes and cutting planes. Preferably, the depth of the slit of the cutting guide is at least 10 mm. This way the direction of the cutting is better controlled, leading to increased accuracy of the resection. For example, the thickness of the cutting guide(s) may be at least 10 mm thick.

In contrast, plastic cutting jigs may be more flexible, leading to reduced cutting accuracy due to e.g. bending and/or generation of sawdust after placement of the cutting jig on the region of interest on the bone surface.

A method of making a patient specific implant for a particular bone segment, such as a segment of a mandible, is as follows. The process may start with a dataset that contains the shape of the bone. That dataset may be generated using, for example, an imaging dataset made with computed tomography (CT) or magnetic resonance imaging (MRI). The imaging dataset may be segmented using a segmentation technique known in the art per se, to create a dataset comprising for example a surface mesh defining a shape of the bone. The dataset may have the format of a stereolithography (STL) file, for example. The dataset may be imported in a 3D editing program, for example a mesh editing program, in which any parts that to not belong to the relevant bone may be removed. The dataset may be cleaned manually or automatically, if necessary by closing any holes and removing any cavities.

Mesh editing tools may be used to reconstruct the shape of the portion of the bone that has been affected by a disease (e.g. a tumor) or a trauma. Also the cuts may be planned to cut away the diseased or otherwise affected portion. The portion of the bone that has to be removed may be deleted from the dataset. Separate datasets may be generated for the implant body and the remaining portion of the bone. These datasets are designed so that they fit together at the planned cuts. For example, the outside surface of the implant body may be shaped so that the implant body fits in between the planned cuts and has planar sections that touch the remaining bone at the cuts

The screw positions may be planned in the remaining healthy bone portion, preferably at least 5 millimeters away from the cut and with at least 7 mm in between adjacent screws. Preferably, the screws are planned perpendicular to the surface of the bone. The flaps may be designed around the screw holes, for example with a minimal thickness of 2 millimeters.

The hollow interior may be designed by (virtually) removing material in the dataset representing the implant, preferably leaving an outer layer of material of at least 0.5 millimeters in thickness. The thickness of the outer layer may be chosen depending on the desired weight of the implant. Any edges of the implant may be rounded.

The design of the hollow interior inside the implant may comprise an open support structure, such as an open mesh structure. Any desired open support structure may be chosen, such as support elements arranged in a rectangular grid or a triangular grid or an irregular grid. The dataset comprising the implant may be stored, for example in the format of an STL file, and provided to a 3D printer for printing.

Preferably, the implant is 3D printed using a biocompatible material as the printing material, preferably a metal, preferably titanium or another biocompatible metal.

Further, a cutting jig may be designed for 3D printing as well. The cuts may be identified in the datasets that were generated in the process of designing the implant. Next the cutting jig may be created with cutting guides corresponding to the planned cuts. The cutting guides may be rigidly connected together by a support structure. Flaps may be added that are similar in shape and location to the flaps of the implant. Preferably, the flaps of the implant may have screw holes with drilling guides at the same place as the screw holes in the flaps of the implant. Preferably the flaps of the cutting jig have further screw holes, preferably on both sides of each drilling guide, so that the cutting jig may be fixed temporarily both to the bone portion that is resected and to the remaining bone portion. Preferably these further screw holes may have a smaller diameter, to support smaller diameter screws, compared to the screw holes corresponding to the screw holes of the implant. Accordingly, the cutting jig may comprise more screw holes than the implant.

Although the implant and method to generate a patient-specific implant, disclosed in this specification, are described in greatest detail in the context of reconstructing the mandible after mandibulectomy, this is not intended to be a limitation of the techniques disclosed herein. On the contrary, the techniques disclosed herein may applied to patient specific implants for any kind of bone reconstructions in the field of orthopedics, including hip reconstructions and knee reconstructions.

In certain embodiments, the implant may be produced by L-PBF (laser powder bed fusion) 3D printing technology. The employed feedstock material may comprise, for example, a metal in powder form, for example titanium, such as Ti6Al4V ELI grade 23.

In certain embodiments, the 3D printing technology enables computer-aided design of the shape of the implant. For example, based on a CT scan an implant may be designed (either automatically or manually), so that the implant replaces the removed piece of bone, e.g. the removed piece of the mandible, and fits exactly to the remaining portion of the mandible. Moreover, by virtue of the design, the shape of the mandible is reconstructed as good as possible, providing an esthetically well looking result.

By creating the implant that fully or sufficiently replaces the removed piece of bone, the use of donor material becomes obsolete. This way, patients that do not have donor material available may be helped. Moreover, by not needing to harvest donor material, the treatment may be less intrusive.

For example, the mass density of mandibular bone may be approximately in the range of 2.0 to 2.2 grams per cubic centimeter. However, in certain patients, such as osteoporosis patients, the mass density of the mandibular bone may be reduced, for example to 1 gram per cubic centimeter.

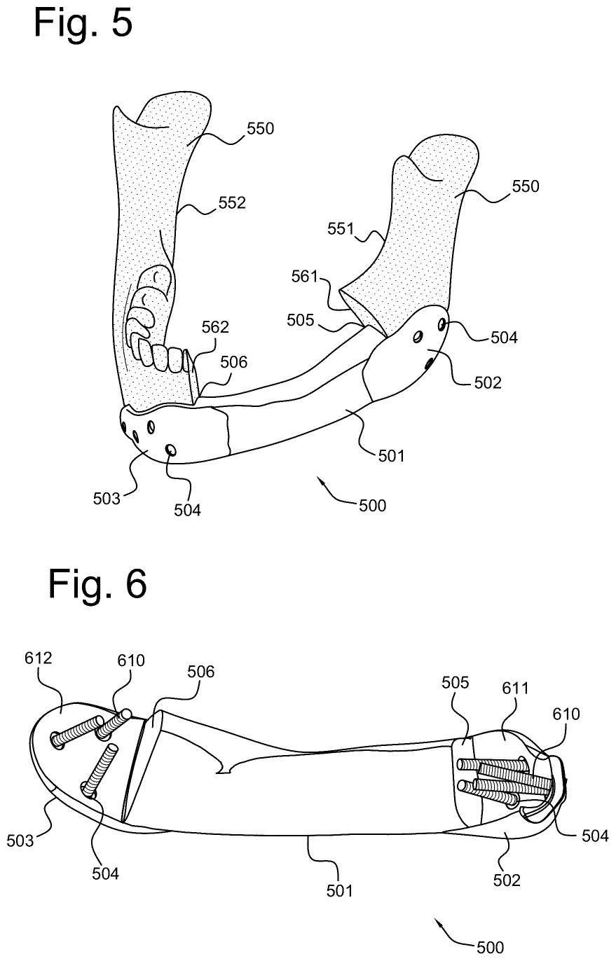

In certain embodiments, the hollow structure of the implant body saves about 45 to 50 percent of mass of the implant, while the implant is still sufficiently strong. For example, titanium has a mass density of about 4.4 grams per cubic centimeter. By reducing to 50% of the mass, a global mass per volume of the implant of about 2.2 grams per cubic centimeter may be reached. In certain embodiments, by making the implant slightly more slender, as in FIG. 5, the weight of the implant may be further reduced. In certain embodiments, the hollow structure of the implant body improves the resistance to tension, so that the implant does not break or bend easily. Compared to other types of implant, the presented implant may be better able to withstand bending forces. The hollow structure of the implant body may improve the strength of the implant body compared to, for example, a non-hollow implant with similar cross sectional surface area. This may, for example, prevent deformation. The strength of the implant, combined with the relatively low weight and the freedom to shape the outside of the implant body as desired, may improve the usability, for example improve occlusion: the ability of a patient to keep the jaws aligned together.

The implant may be hollow. This reduces the weight of the implant, and allows the weight to be close to the original bone, even though a relatively heavy material (e.g. titanium) can be used for the implant. For improved strength the hollow implant may have an internal support structure, preferably a mesh structure.

The improved strength of the implant reduces the likelihood of undesired deformations or fractions. By virtue of the better positioning of the screws and the stronger design, forces caused by e.g. chewing may be distributed over the screws in an enhanced manner. This may avoid loosening of the screws. For example, self-locking screws may be employed. Moreover, in certain embodiments, irritations in the surrounding tissues may be avoided or reduced by virtue of the anatomical shape of the implant and the improved distribution of the forces on the surrounding tissues.

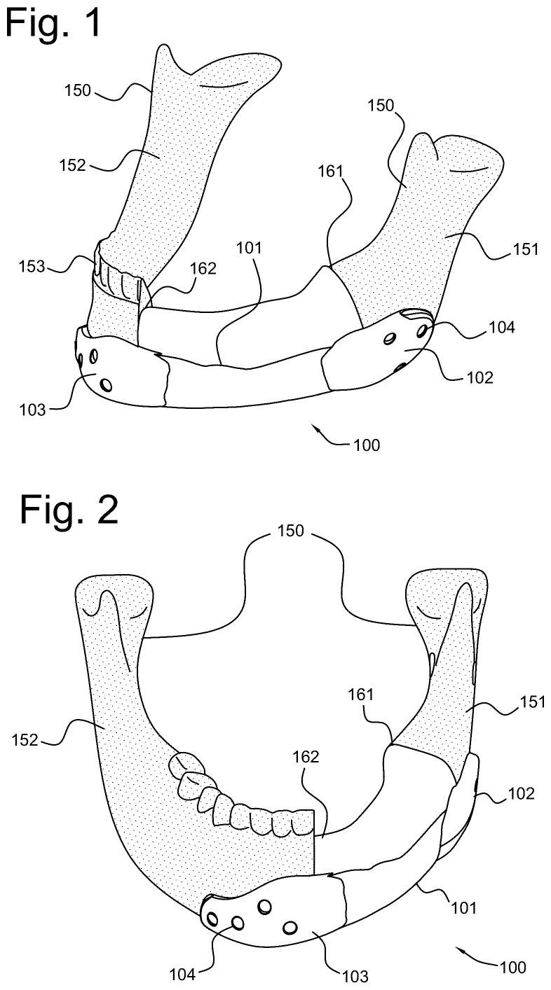

FIG. 1 shows an oblique side perspective view of an example of an implant 100 after implantation in the mandible 150. FIG. 2 shows a frontal perspective view of the implant 100 in the context of the mandible 150, as shown in FIG. 1. These drawings show the mandibular implant 100 with a view of the implant body 101 and flaps 102, 103. The drawings further show a sketch of the remaining parts 151, 152 of the mandible 150. As shown in the drawings, the mandible 150 has been resected, leaving a first part 151 and a second part 152 of the mandible 150. The implant body 101 fits in the space of the resected bone, between the cutting plane 161 of the first remaining part 151 of the mandible 150 and the cutting plane 162 of the second remaining part 152 of the mandible 150. A number of screw holes 104 is provided in both flaps 102, 103. As can be appreciated from FIG. 1 and FIG. 2, the shape of the implant body 101 is curved to best reconstruct the mandible. The height of the implant body 101 is approximately similar to the height of the mandible 150 The flaps 102, 103 extend from the implant body in a direction to fit to the remaining parts of the mandible.

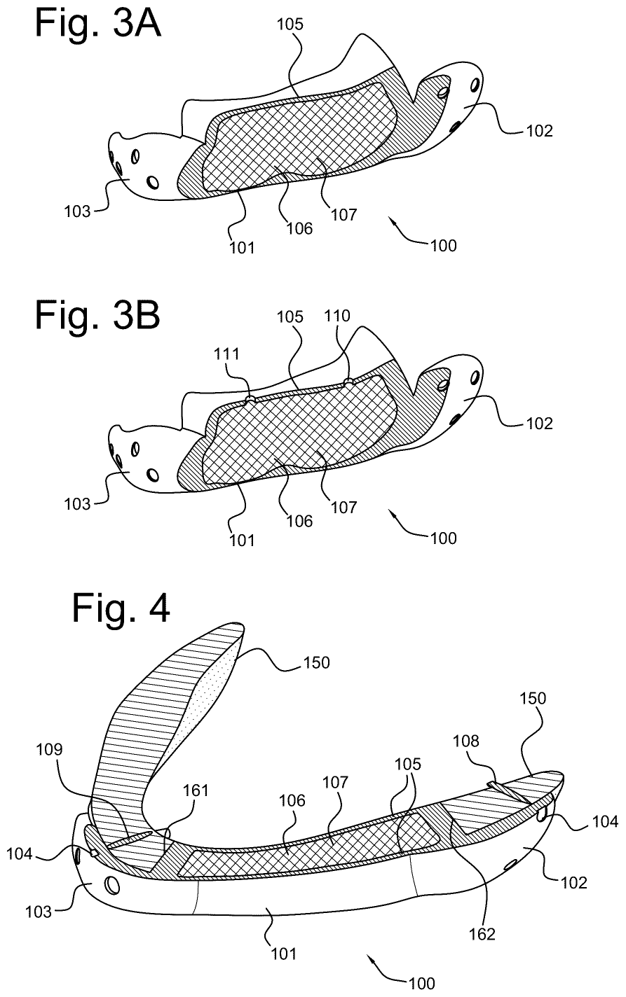

FIG. 3A and FIG. 4 show partly worked open views of the implant of FIG. 1 and FIG. 2. FIG. 4 also shows the bone 150 to which the implant 100 is connected. As shown in FIG. 4, the implant body 101 comprises a solid layer 105 and a cavity 106. The cavity 106 comprises a mesh structure 107. The solid layer 105 and the mesh structure 107 may be made of the same material. The solid layer 105 fully encloses the cavity 106, so that the cavity 106 is closed and sealed, so that there is no contact or fluid exchange between the cavity 106 and the surrounding tissues or bone. For example, the cavity 106 may contain air. As shown in FIG. 4, screws 108 and 109 extend through the screw holes 104 and the bone of the remaining mandala 150.

FIG. 3B shows a section view of the mandibular implant 100, in a production stage according to an embodiment. In certain embodiments, as illustrated in FIG. 3A, one or more holes 110, 111 are created in the solid layer 105. For example, air may be forced in to the inner cavity 106 through a first hole 110 in the solid layer 105 by applying air pressure, while air is allowed to be released through at least one other hole 111 in the solid layer. This way, any dust or feedstock material may be removed from the inner cavity 106 after its creation. After this cleaning procedure, the one or more holes 110, 111 may be permanently sealed, as illustrated in FIG. 3A.

FIG. 5 shows a different example of an implant 500 in the context of the mandible 550. Compared to the implant 100, the height of the implant body 501 is less than the height of the mandible 550. This way, the planar sections 505, 506 of the implant body may cover a portion of each cutting plane 561, 562 of the remaining bone. For the remainder, the features of the implant 500 are similar to the features of the implant 100. The implant 500 may be stably positioned in between the cutting planes 561 and 562, further reinforced by the flaps 502, 503.

FIG. 6 shows a perspective view of the implant 500 of FIG. 5, by itself. In the shown example, the surface of the implant body 510 has two planar sections 505, 506 on two opposite sides of the implant body 501. In between the planar sections, the shape may be designed to support the shape of the desired reconstruction. For example, the implant body 501 may be curved in between the two planar sections, in accordance with the portion of the bone that it serves to replace. From the edge of the planar sections 505, 506, flaps 502, 503 extend. The surface 611 of the flap 502 on the side of the planar section 505 may have a shape that matches the shape of the bone portion on which the flap 502 lies, next to the cutting plane 561. The surface 612 of the flap 503 on the side of the planar section 506 may have a shape that matches the shape of the bone portion on which the flap 503 lies. Screws 610 are schematically drawn that extend through the screw holes 504. The screws 610 penetrate the remaining bone 551, 552, under the flap. In certain embodiments, the orientation of the screws 610 may be substantially perpendicular to the surface of the flap 502 on the side of the cutting plane 505. In other words, the orientation of a screw 510 may be substantially perpendicular to the surface of the bone which the screw penetrates.

In certain embodiments, an angle between the surface 611 of the flap 502 and the planar section 505 of the surface of the implant body 501 may be less than 90 degrees. This may hold for any one of the flaps 502, 503. A smaller angle between flap and planar section may help to better clamp the implant in place. This angle may be smaller than 90 degrees for all flaps or just some of the flaps.

In certain embodiments, the implant body may comprise one or more placeholders for dental implants. These dental implants may be attached to these placeholders, possibly after the implantation of the implant is complete. Such a placeholder may comprise a slot or hole, in which the dental implant may be fixated.

Further, the placeholder may comprise a clamping mechanism or screw thread to facilitate the fixation of the dental implant. Alternatively, the dental implants may be attached to the implant body before the implantation. Yet alternatively, the dental implants are integrated with the implant body.

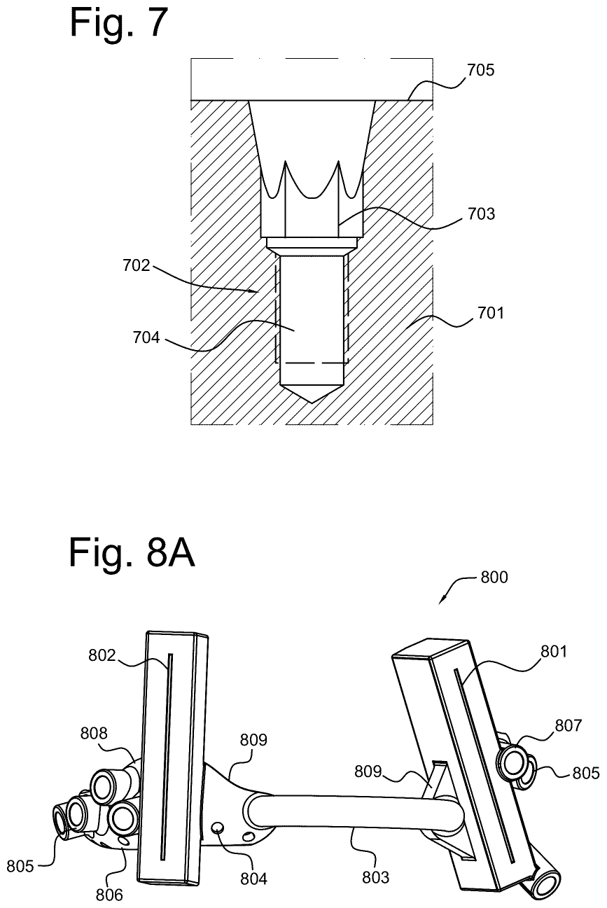

FIG. 7 shows an example of a placeholder 702 for a dental implant in the solid layer 701 of the implant body. For example, the solid layer 701 may be made locally thicker to provide sufficient space for the dental implant placeholder 702. As illustrated, the placeholder 702 may comprise a bolt 703 below the outside surface 705 of the implant body. The bolt may be secured to the solid layer 701 of the implant body by an anchor 704. It will be understood that other constructions to support dental implants may be provided instead.

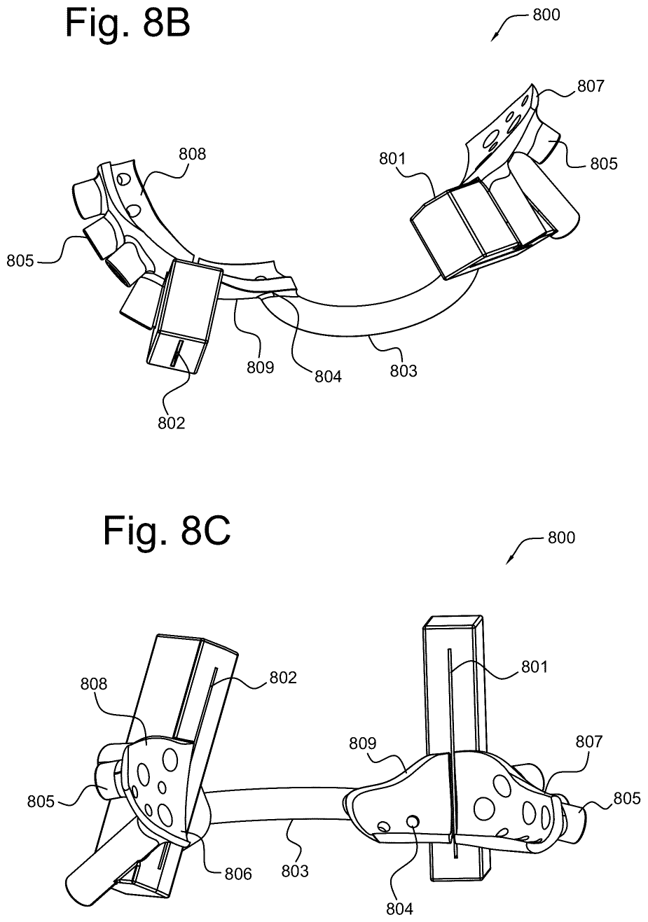

FIG. 8 shows several perspective views of a patient specific cutting jig 800, or sawing jig. Specifically, FIG. 8A shows a front view, FIG. 8B shows a top view, and FIG. 8C shows a back view. The cutting jig 800 comprises one rigid structure that can be fixed to the bone of the patient before making any cuts. The cutting guides 801, 802 define the cutting planes for the cutting procedure. The relative position and orientation of these cutting planes correspond to the planar sections of the implant body. To that end, the cutting guides 801, 802 are connected by a rigid bridge 803. This way, after cutting, the implant body may be clamped in between the cutting planes. The orientation of the flaps of the implant may further clamp the implant to stabilize its position.

The cutting jig 800 may comprise flaps 807, 808 extending from the cutting guides 801, 802. Flaps 807, 808 may comprise screw holes 805 that match the position of the screw holes in the flaps of the implant. These screw holes 805 may be provided with conical or cylindrical drilling guides. That way, the holes in the bone may be prepared in the correct orientation, so that the screws in through the flaps of the implant itself will thereafter also assume that orientation.

The cutting jig 800 may comprise screw holes 804, 806 on both sides of the cutting guide 802 to temporarily fix the cutting jig 800 to both the bone to be resected and the remaining bone portion. These screw holes 804, 806 may be smaller than the screw holes 805. In certain embodiments, the screw holes 804 on the side of the cutting guide 802 opposite the screw holes 805 may be provided while the smaller screw holes 806 on the side of the screw holes 805 may be omitted. In certain embodiments, the screw holes 806 are placed in flap 808 and the screw holes 804 are placed in a flap 809 on the side of the cutting guide 802 opposite of the flap 808. These smaller screw holes 804, 806 may be useful to initially fix the cutting jig 800 to the bone and/or to keep the resected bone in place fixed to the cutting jig 800.

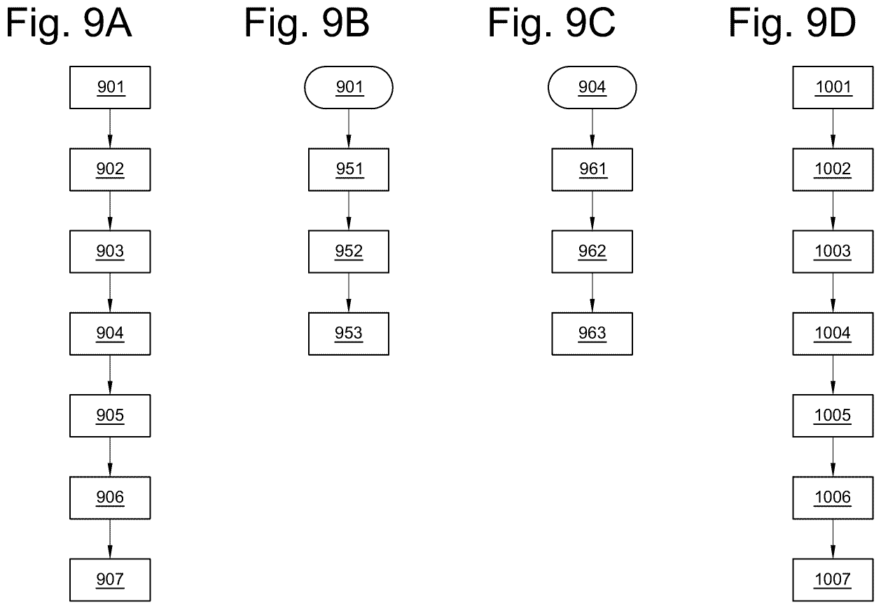

FIG. 9A shows a flowchart illustrating aspects of a method of creating a personalized implant.

In step 901, a shape of an implant body is determined, based on an anatomical shape of a bone represented by an image dataset. For example, the image dataset can be one or more of a computed tomography (CT) image dataset, a magnetic resonance imaging (MRI) dataset, or any other acquired image dataset representing the relevant part of the bone of a patient and allows for digital 3D reconstruction of the bone structure. For example, the output of step 901 may comprise a dataset representing the shape of the implant body. Such a dataset may have the format of a stereolithography dataset, for example, although other formats can be used alternatively. The determined shape of the implant body includes the interior shape of the implant body, which includes a closed cavity. This closed cavity is fully enclosed by a solid layer. This solid layer forms an outside surface exposed to the exterior of the implant.

FIG. 9B shows an example implementation of step 901. In step 951, the shape model of the bone of a patient is obtained, from e.g. a CT scan or MRI scan. In step 952, at least one cutting plane is determined relative to the bone shown in the shape model. This cutting plane may be a plane where it is desired to cut the bone for resection. For example, two cutting planes may be defined to remove a portion of bone in between the two cutting planes. In another example, one cutting plane may be defined to remove a portion of bone on one side of the cutting plane. Alternatively, more cutting planes may be defined to cut out a portion of bone. In step 953, the shape of the implant body is determined based on the shape model and the at least one cutting plane. The outside surface of the implant body may have at least one planar section corresponding to at least one cutting plane. For example, two planar sections correspond to two cutting planes. The relative position and orientation of these two planar sections corresponds to the two cutting planes. This may ensure a good fit. Moreover, in between the two planar sections the surface may be smoothly curved to mimic an organic anatomical bone portion.

Referring again to FIG. 9A, in optional step 902, one or more holes may be designed in the solid layer. These holes fluidly connect the inner cavity to the exterior of the implant, until the one or more holes are sealed.

In optional step 903, an open support structure, such as a mesh structure, may be designed to fill up the closed cavity. This support structure may be added to the dataset that represents the shape of the implant body. The support structure may comprise a structure of threads of material arranged in a 3D grid, such as a 3D rectangular grid or a 3D triangular grid.

In optional step 904, at least one flap may be added to the shape model representing the shape of the implant. For example, these flaps may be manufactured integrally with the implant body during the subsequent additive manufacturing step. Alternatively, the at least one flap may be manufactured separately and attached to the implant body in a later step (not illustrated), for example by molding.

FIG. 9C illustrates a process of designing the at least one flap. In step 961, a suitable location at the edge of the cutting plane is determined based on the shape model of the bone. In step 962, the orientation and/or curvature of the at least one flap may be designed using the shape model of the bone, to correspond to a surface of the bone adjacent the cut. That way, when the implant is used in practice, the flap can cover a bone portion adjacent the cut. In step 963, one or more screw holes may be designed in the at least one flap. In a particularly advantageous embodiment, at least three screw holes are created in each flap, wherein the at least three screw holes are arranged in a non-linear pattern in order to distribute the load baring forces more equally over the screws.

Referring again to FIG. 9A, in step 905, the implant body may be created according to the determined shape. This step may be implemented by means of additive manufacturing or 3D printing, using the dataset representing the shape of the implant body as the basis. The material used to create the solid layer of the implant body may be non-transmissive for fluids, so that the closed cavity is fluidly isolated from the exterior of the implant. The material used may further be rigid. For example, a metal such as titanium may be used as the material for at least the solid layer and the support structure or mesh structure.

In optional step 906, if the one or more holes have been made in the solid layer, these holes may be used to clean the inner cavity of the implant body. For example, air pressure may be applied to at least one hole, thus causing any feedstock remaining inside the inner cavity to be blown out of the inner cavity through the same or another hole.

In step 907, if one or more holes have been made in the solid layer, these holes may be sealed, so that the solid layer is closed and the inner cavity becomes fully enclosed by the solid layer. For example, the one or more holes may be sealed using the same material used for the solid layer, by bonding or welding for example. After this, the outside surface may be levelled. In addition, or alternatively, any suitable surface treatment, such as polishing and/or coating, may be applied to the surface of the implant body.

It will be understood that other methods of manufacturing the implant may be considered. For example, the support structure may be manufactured separately and shaped according to the shape of the inner cavity, After that, the solid layer may be created from a number of plates that may be placed around the support structure and molded together and polished to achieve a desired smooth shape. The flaps may be manufactured by e.g. welding, and be attached to the implant body by e.g. molding.

FIG. 9D shows a flowchart illustrating aspects of a method of manufacturing a cutting jig. In step 1001, a shape model of the bone of the patient is obtained. This shape model may be similar to the shape model obtained in step 901. In step 1002, at least one cutting plane is determined, intersecting the bone. The at least one cutting plane may correspond to the at least one cutting plane determined in step 952. In step 1003, a cutting guide is designed according to the at least one cutting plane. In case a plurality of cutting guides is designed for a plurality of cutting planes, step 1003 may comprise designing a support structure to fix the relative position between different ones of the plurality of cutting guides in accordance with the relative positions of the cutting planes. This way, the relative positions of the cutting planes defined by the cutting guides may correspond to the relative positions of the planar sections of the implant body generated, for example, by the method of FIG. 9.

In step 1004, at least one cutting guide is provided with a flap having an orientation, location, and/or curvature corresponding to the orientation, location, and/or curvature of the flap of the planar section of the implant body corresponding to the at least one cutting guide.

In step 1005, one or more screw holes may be designed in the flap of the cutting jig, these one or more screw holes may be provided at the same location as in the flap of the implant. These screw holes of the cutting jig may be provided with screw guides to guide the direction of the screw.

In step 1006, one or more flaps may be designed on the side of the cutting guide opposite the flap designed in step 1005. These flaps may also be provided with one or more screw holes.

In step 1007, the cutting jig with the cutting guide, support structure, and flap is manufactured based on the created design, by means of additive manufacturing or 3D printing. It will be understood that the cutting jig may be manufactured by a different method, e.g. by forging or molding.

Implants designed and manufactured using the techniques disclosed herein may lead to an improved aesthetic outcome. Compared to for example bone graft reconstruction, the result may look more natural.

The techniques disclosed herein may allow to provide a highly accurate fit to the patient's bone after resection. This feature, in combination with the relatively high bending resistance of the implant, may prevent breaks and deformations of the implant, and may lead to accurate long-lasting reconstruction. In case of mandibular reconstruction, it may lead to good occlusion.

The implant may connect to the bone at the saw plane and the flap which is matched to the bone. This construction allows for a tight and firm connection.

The flaps allow for out of line, i.e. triangular, screw placement. This is a mechanically advantageous placement.

The concave shape of the flap also results in a relatively high stiffness, which allows forces on the implant to be more evenly distributed over the screws. This may help to mitigate the risk of screw breakout or failure.

The relatively large surface area of the implant without sharp edges (compared to, for example, a strip implant) reduces local pressure points on the surrounding tissue. The techniques disclosed herein may allow to integrate dental implants. This may help to reconstruct the teeth of the patient.

An optional integrated mesh structure provided on the interface between the implant and the bone may allow for osseointegration.

A metal jig is more rigid compared to commonly used plastic jigs. This allows for a more accurate resection. Moreover, plastic saw guides can produce chips when in contact with the saw, which can result in chips ending up in the patient tissue. This may be avoided by using the metal jig.

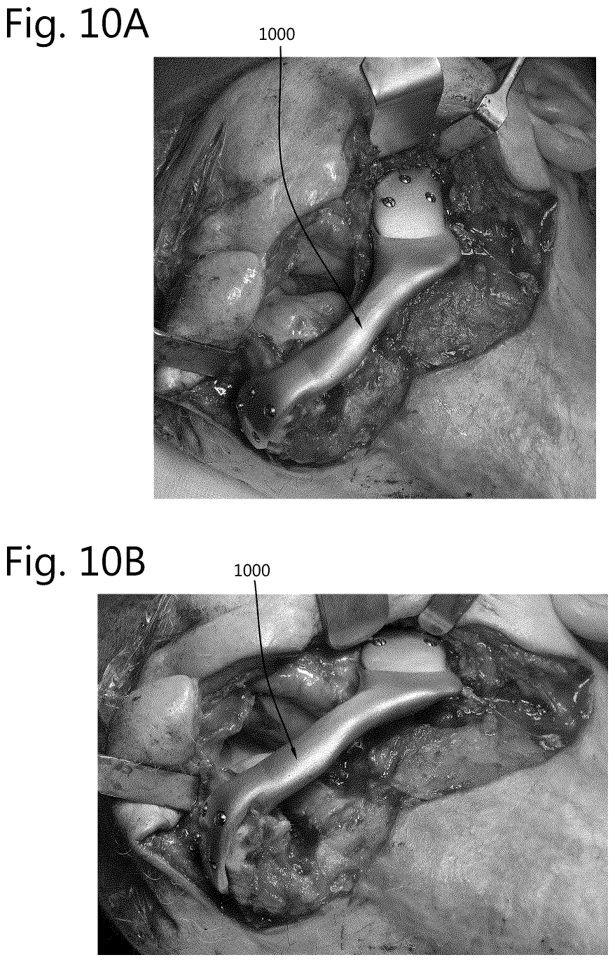

FIG. 10A and FIG. 10B show a patient case example of the mandibular implant replacing the removed bone and reconnecting the remaining bone segments.

Some or all aspects of the invention may be suitable for being implemented in form of software, in particular a computer program product. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

Aspects of the invention are disclosed in the following clauses.

Clause 1. A personalized implant for bone replacement, comprising an implant body comprising a closed cavity and a solid impervious layer of material defining an outside surface of the implant body exposed to an exterior of the implant, the solid impervious layer fully enclosing the closed cavity inside the implant body.

Clause 2. The implant of clause 1, wherein the closed cavity comprises an open support structure to reinforce the solid impervious layer.

Clause 3. The implant of clause 1, wherein the outside surface of the implant body is piecewise smooth.

Clause 4. The implant of clause 1, wherein the outside surface comprises at least one planar section.

Clause 5. The implant of clause 4, wherein the outside surface comprises at least two planar sections, arranged on opposite ends of the implant body.

Clause 6. The implant of clause 4, further comprising at least one flap extending from the implant body at an edge of the at least one planar section of the outside surface.

Clause 7. The implant of clause 6, wherein the at least one flap has a concave surface.

Clause 8. The implant of clause 6, wherein at least one of the flaps comprises at least three screw holes arranged in a pattern of at least one triangle.

Clause 9. The implant of clause 1, wherein a weight of the implant divided by a volume of the implant is at most 2.3 grams per cubic centimeter, and a density of the solid layer is at least 4 grams per cubic centimeter.

Clause 10. The implant of clause 1, wherein the implant further comprises an outside mesh structure on the outside surface, wherein the outside mesh structure is exposed to an exterior of the implant.

Clause 11. A method of producing a personalized implant for bone replacement, the method comprising
determining a shape of an implant body based on an anatomical shape of a bone represented by an image dataset;
creating the implant body according to the determined shape,
wherein the implant body comprises a closed cavity and a solid impervious layer of material defining an outside surface of the implant body exposed to an exterior of the implant, the solid impervious layer fully enclosing the closed cavity inside the implant body.

Clause 12. The method of clause 11, wherein the closed cavity comprises an open support structure to reinforce the solid impervious layer.

Clause 13. The method of clause 11,
wherein the creating the implant body comprises creating the solid impervious layer with at least one hole in the solid impervious layer, wherein the at least one hole fluidly connects the cavity with an exterior of the implant body, and the method further comprises:
removing feedstock material from the cavity using the at least one hole; and
sealing the at least one hole to fluidly close the cavity.

Clause 14. The method of clause 11, wherein the determining the shape of the implant body comprises
obtaining a shape model of the bone of the patient;
determining at least one cutting plane intersecting the bone; and
determining the shape of the implant body based on the shape model and the at least one cutting plane,
wherein the outer surface of the solid layer of the implant body comprises at least one planar section corresponding to the at least one cutting plane.

Clause 15. The method of clause 14, wherein the method further comprises creating at least one flap extending from the implant body at an edge of the at least one planar section corresponding to the at least one cutting plane.

Clause 16. The method of clause 15, wherein the method further comprises determining, based on the shape model, an orientation or curvature of the at least one flap relative to the cutting plane, to match an orientation or curvature of a surface of the bone of the patient adjacent the at least one cutting plane.

Clause 17. The method of clause 15, further comprising creating at least three screw holes in the at least one flap, wherein the at least three screw holes are arranged in a pattern of at least one triangle.

Clause 18. The method of clause 15, further comprising creating a cutting jig having at least one cutting guide and at least one flap, wherein the at least one cutting guide defines the at least one cutting plane and wherein a shape, orientation, and location of the at least one flap of the at least one cutting jig with respect to the at least one cutting plane matches at least part of a shape, an orientation, and a location of the at least one flap extending from the implant body.

Clause 19. The method of clause 18, further comprising creating screw holes in the flap of the cutting jig, wherein the screw holes are provided with drilling guides and wherein the screw holes match corresponding screw holes in the flap of the implant.

1. A personalized implant for bone replacement, comprising an implant body as a reconstruction of a bone portion, wherein the implant body comprises a closed cavity and a solid impervious layer of material defining an outside surface of the implant body exposed to an exterior of the implant, the solid impervious layer fully enclosing the closed cavity inside the implant body.

2. The implant of claim 1, wherein the closed cavity comprises an open support structure to reinforce the solid impervious layer.

3. The implant of claim 1, wherein the outside surface of the implant body is piecewise smooth.

4. The implant of claim 1, wherein the outside surface comprises at least one planar section.

5. The implant of claim 4, wherein the outside surface comprises at least two planar sections, arranged on opposite ends of the implant body.

6. The implant of claim 4, further comprising at least one flap extending from the implant body at an edge of the at least one planar section of the outside surface.

7. The implant of claim 6, wherein the at least one flap has a concave surface.

8. The implant of claim 6, wherein at least one of the flaps comprises at least three screw holes arranged in a pattern of at least one triangle.

9. The implant of claim 1, wherein a weight of the implant divided by a volume of the implant is at most 2.3 grams per cubic centimeter, and a density of the solid layer is at least 4 grams per cubic centimeter.

10. The implant of claim 1, wherein the implant further comprises an outside mesh structure on the outside surface, wherein the outside mesh structure is exposed to an exterior of the implant.

11. A method of producing a personalized implant for bone replacement, the method comprising
determining a shape of an implant body as a reconstruction of a bone portion based on an anatomical shape of a bone, wherein the implant body comprises a closed cavity and a solid impervious layer of material defining an outside surface of the implant body exposed to an exterior of the implant, the solid impervious layer fully enclosing the closed cavity inside the implant body; and
creating the implant body according to the determined shape.

12. The method of claim 11, further comprising designing an open support structure inside the closed cavity to reinforce the solid impervious layer.

13. The method of claim 11,
wherein the creating the implant body comprises creating the solid impervious layer with at least one hole in the solid impervious layer, wherein the at least one hole fluidly connects the cavity with an exterior of the implant body, and the method further comprises:
removing feedstock material from the cavity using the at least one hole; and
sealing the at least one hole to fluidly close the cavity.

14. The method of claim 11, wherein the determining the shape of the implant body comprises
obtaining a shape model of the bone of the patient;
determining at least one cutting plane intersecting the bone; and
determining the shape of the implant body based on the shape model and the at least one cutting plane,
wherein the outer surface of the solid layer of the implant body comprises at least one planar section corresponding to the at least one cutting plane.

15. The method of claim 14, wherein the method further comprises creating at least one flap extending from the implant body at an edge of the at least one planar section corresponding to the at least one cutting plane.

16. The method of claim 15, wherein the method further comprises determining, based on the shape model, an orientation or curvature of the at least one flap relative to the cutting plane, to match an orientation or curvature of a surface of the bone of the patient adjacent the at least one cutting plane.

17. The method of claim 15, further comprising creating at least three screw holes in the at least one flap, wherein the at least three screw holes are arranged in a pattern of at least one triangle.

18. The method of claim 15, further comprising creating a cutting jig having at least one cutting guide and at least one flap, wherein the at least one cutting guide defines the at least one cutting plane and wherein a shape, orientation, and location of the at least one flap of the at least one cutting jig with respect to the at least one cutting plane matches at least part of a shape, an orientation, and a location of the at least one flap extending from the implant body.

19. The method of claim 18, further comprising creating screw holes in the flap of the cutting jig, wherein the screw holes are provided with drilling guides and wherein the screw holes match corresponding screw holes in the flap of the implant.
